(19) United States
(12) Patent Application Publication
(10) Pub. No.: US 2007/0191368 A1
(43) Pub. Date: Aug. 16, 2007

(54) Inhibition of tissue damage to skin from radiation treatment therapy

(76) Inventors: Donald L. Bissett, Hamilton, OH (US); James J. Nordlund, Cincinnati, OH (US)

Correspondence Address: MILES & STOCKBRIDGE PC, 1751 PINNACLE DRIVE, SUITE 500, MCLEAN, VA, 22102-3833, US

(21) Appl. No.: 10/806,474

(22) Filed: Mar. 23, 2004

(51) Int. Cl. A61K 31/53; A61K 31/497; A61K 31/4965; A61K 31/4709; A61K 31/401; A61K 31/444; A61K 31/35

(52) U.S. Cl. 514/241; 514/252.11; 514/255.05; 514/314; 514/332; 514/345; 514/471; 514/566; 514/423; 514/640; 514/569; 514/460; 514/397; 514/553

(57) ABSTRACT

A method of and composition for inhibiting the deleterious effects on mammalian skin tissue by radiation treatment therapy comprising administering to a patient in need thereof, in conjunction with exposure of the skin tissue to radiation, a safe and effective amount of a chelating agent.

### FIELD OF THE INVENTION

This invention relates to compositions, methods and articles of manufacture useful for protecting mammalian tissue from the harmful effects of radiation treatment therapy.

### BACKGROUND OF THE INVENTION

Radiation therapy involves the treatment of diseased, e.g., cancerous tissue with high-energy radiation according to a treatment regimen. The treatment regimen controls the radiation's placement and dose level so that the diseased tissue receives a sufficient dose of radiation while the radiation to surrounding and adjacent non-diseased tissue is minimal.

Intensity modulated radiation therapy (IMRT) treats a patient with multiple rays of radiation each of which may be independently controlled in intensity and/or energy. The rays are directed from different angles about the patient and combine to provide a desired dose pattern. Typically, the radiation source consists of either high-energy X-rays, electrons from certain linear accelerators, or gamma rays from highly focused radioisotopes such as Co⁶⁰.

Methods of producing intensity modulated rays of radiation are well known in the art and include the stop and shoot method, (Xia, P., Verhey, L. J., “Multileaf Collimation Leaf Sequencing Algorithm for Intensity Modulated Beams with Multiple Static Segments,” Medical Physics, 25:1424-34 (1998)), the sliding window technique (Bortfeld, et al., “Realization and Verification of Three-Dimensional Conformal Radiotherapy With Modulated Fields,” Int'l J. Radiat. Oncol. Biol. Phys., 30:899-908 (1994)), intensity modulated arc therapy, (Yu, C. X., “Intensity-Modulated Arc Therapy With Dynamic Multileaf Collimation: An Alternative to Tomotherapy,” Physics in Medicine & Biology, 40:1435-49 (1995)), and sequential (axial) tomotherapy, (Carol, et al., “The Field-Matching Problem as it Applies to the Peacock Three Dimensional Conformal System for Intensity Modulation,” Int'l J. Radiat. Oncol. Biol. Phys., 34:183-87 (1996)).

There are long-term hazards associated with such radiation exposure. One of these long-term hazards is malignant changes in the skin surface. Numerous epidemiological studies demonstrate a strong relationship between radiation exposure and human skin cancer. Another long-term hazard of such radiation is premature aging of the skin. This condition is characterized by wrinkling and yellowing of the skin, along with other physical changes such as cracking, telangiectasis (spider vessels), solar keratoses (growths), ecchymoses (subcutaneous hemorrhagic lesions), and loss of elasticity (sagging). These hazards are believed to be caused by the free radicals generated by the radiation. For example, when tissues are exposed to gamma radiation, most of the energy deposited in the cells is absorbed by water and results in scission of the oxygen-hydrogen covalent bonds in water, leaving a single electron on hydrogen and one on oxygen creating two radicals H and OH. The hydroxyl radical, OH, is one of the most reactive known in chemistry. It reacts with biomolecules and sets off chain reactions and can interact with the purine or pyrimidine bases of nucleic acids. Indeed, radiation-induced carcinogenesis may be initiated by free radical damage (Breimer L H (1988) Brit. J. Cancer 57: 6).

To date, there has been little research on ways to mitigate the effects of high-energy radiation exposure on the skin.

It is an object of the present invention to provide a composition in a stable form, the use of which will prevent or inhibit the damaging effects of exposure to high-energy radiation employed in radiation treatment therapy programs.

It is also an object of the present invention to provide a method for preventing or inhibiting the damaging effects of exposure to high-energy radiation employed in radiation treatment therapy programs.

It is further an object of the present invention to provide an article of manufacture for the prevention or inhibition of the damaging effects of exposure to high-energy radiation employed in radiation treatment therapy programs.

### SUMMARY OF THE INVENTION

One embodiment of the invention relates to a method of inhibiting the deleterious effects of radiation treatment therapy on skin tissue comprising administering a chelating agent to a mammal (human or non-human) in conjunction with, prior to or after exposing the patient to radiation treatment therapy. The method of the invention includes administering the chelating agent topically, orally or intravenously.

Another embodiment of the invention concerns a radiation treatment protective composition comprising:
(a) a safe and radioprotectively effective amount of a chelating agent; and
(b) a safe and effective amount of a suitable carrier therefore, and, optionally, a safe and radioprotectively effective amount of an anti-inflammatory agent and/or a free radical scavenging agent (e.g., an anti-oxidant).

A further embodiment of the invention comprises an article of manufacture comprising packaging material and a chelating agent contained within the packaging material, wherein the chelating agent is effective for inhibiting the deleterious effects of radiation treatment therapy on skin tissue, and wherein the packaging material comprises a label which indicates that the chelating agent can be used for protecting against the deleterious effects of radiation treatment therapy on skin tissue.

The present invention also relates to methods of and compositions and articles of manufacture for inhibiting the deleterious effects of radiation treatment therapy on skin tissue based on the administration of safe and effective amounts of a specifically selected chelating agent to a mammal in need thereof in combination with any or all of the following: a safe and effective amount of an anti-inflammatory agent, and a safe and effective amount of a free radical scavenging agent (e.g., an anti-oxidant), the combination being administered in conjunction with, prior to or after exposing the skin to radiation treatment therapy.

### DETAILED DESCRIPTION OF THE INVENTION

Active Agent

It is known that high-energy radiation induces harmful reactions in skin. During exposure and as repair of the damage takes place, super-oxide (O₂⁻) radicals are formed in the skin. High-energy radiation also causes some microvascular damage in the skin. This leads to local hemorrhage and “leakage” of blood cells into the dermis and/or iron-containing proteins (e.g., transferring). Iron from the hemoglobin accumulates in the extra-cellular matrix of the tissue as Fe⁺² and Fe⁺³. It is known that iron catalytically participates in the conversion of superoxide radicals to hydroxyl radicals, a species which is known to be very damaging to tissue. (See Davies, J. Biol. Chem., Vol. 262, No 20 (1987), pp. 9895-9901). Another process which is damaging to tissue is membrane lipid peroxidation, which is also accelerated by iron. (See Holliwell and Gutteridge, Free Radicals in Biology and Medicine, Claredon Press, Oxford, England (1985), p. 147). It is believed that this damage results in premature aging of the skin. The deposited iron may also be taken up by viable skin cells. This can result in iron-catalyzed DNA damage within the cells which may ultimately cause the formation of tumors or abnormal matrix components in the skin. It is also known that other metal ions such as Cu⁺¹ or Cu⁺² can participate catalytically in the generation of oxygen radicals and that such ions may also result in skin damage.

Without being bound by theory, it has now been found that certain metal chelators, which are able to “tie-up” free iron as well as other metals, thus impairing their catalytic activity, to protect the skin from the damaging effects caused by high-energy radiation exposure.

Not all chelating agents are useful in the present invention. Chelating agents which may be used herein must be safe for administration and exhibit the required protective effect. By “safe” is meant chelating agents which may be used topically, orally or intravenously at typical usage levels for extended periods of time, without causing any significant adverse skin reactions or other side effects. One in vitro test for determining whether a chelating agent might exhibit a radioprotective effect is described below. It is to be understood, however, that passage of the below-described test is not a requisite for the chelating agents of the present invention.

Test Method

In Vitro Solution Radical Assay

This assay may be used to identify chelators that have the ability to scavenge or to inhibit hydroxyl radical formation. The assay is based on the OH oxidation of methional to ethylene. The efficacy of any given chelator is determined from its ability to inhibit ethylene formation.

More specifically, the method (based on Zigler et al., Arch Biochem. Biophys. 241(1), 163-172 (1985) and Tauber et al., J. Clin. Invest, 60, 374-379 (1977)) is carried out as follows. The following materials are added to a one dram vial: 200 μl phosphate buffered saline (PBS), 100 ul of 1.4 mM hypoxanthine in PBS, 100 ul of 1.0 mM FeSO.sub.4 in PBS, 100 μl of 3.0 mM EDTA in PBS, molecule to be tested, 100 μl of 3.5 mM methional in PBS, 100 μl xanthine oxidase (0.1 unit of enzyme/ml). The reaction vial is then capped with Self-Seal Septa Assembled in Vial Cap (Waters Associates, Cat. #73010), gently mixed on a Vortex Genie, and incubated at 37° C. for 20 min. in an Aquatherm Water Bath Shaker (New Brunswick Scientific). At this time the reaction is stopped by placing all samples at −10° C. in a Lauda RM-20 constant temperature bath. One 500 μl aliquot (Pressure lock syringe, Precision Sampling Corporation) is then injected into a Gas Chromatograph (HPO-5880 A) fitted with a chromosorb 102 column (10 ft.×⅛” O.D.) at an oven temperature of 35° C. using helium as the carrier gas at 30 ml/min. Triplicate preparations are run for each scavenger tested; the control contains no enzyme (no ethylene should be produced). Calibration of the Gas Chromatograph is done by injecting various volumes of ethylene gas (Supelco #2-2572) of known concentration and correlating the amount injected with total peak area. An ethylene standard is run for each set of experiments.

The % inhibition of hydroxyl radical formation is determined by the following method: First, all peak areas from the chromatograms are converted into picomoles (p moles) of ethylene using a calibration curve for ethylene. Then, according to the equation:
[(pmoles C₂H₄ w/o scavenger-pmoles C₂H₄ with scavenger)/pmoles C₂H₄ w/o scavenger]×100=% Inhibition.

Chelating agents which exhibit at least about a 50% inhibition of iron-catalyzed hydroxyl radical formation in this assay are useful in the present invention. The chelating agents useful in the present invention may be classified according to their donor groups; see Martell, A. E., “The Design and Synthesis of Chelating Agents”, Development of Iron Chelators for Clinical Use, Martell, Anderson and Badman, eds., Elsevier North Holland, Inc., New York, N.Y. (1981), pp. 67-104, which is hereby incorporated by reference. Because some chelating agents have more than one type of donor group, they may fall into more than one of the classes defined in Martell.

Preferred classes containing chelating agents useful in the present invention are aromatic armine, carbonyl, oximate, amine, carboxylate, alkoxide, enolate, phenoxide, catecholate, hydroxy acid, hydroxamate, ketoenolate, mercaptide, hydroxy aromatic amine, polyphosphates and aromatic hydroxy acid. More preferred classes are aromatic amine, carbonyl, oximate, enolate, phenoxide, catecholate and hydroxylate; especially preferred are aromatic amine, carbonyl, oximate and enolate.

Preferred chelating agents useful in the present invention which fall within the class of aromatic amines include the following: 2,2′-dipyridylamine; 1,10-phenanthroline {o-phenanthroline}; di-2-pyridyl ketone; 2,3-bis (2-pyridyl) pyrazine; 2,3-bis (2-pyridyl)-5,6-dihydropyrazine; 1,1′-carbonyldiimidazole; 2,4-bis (5,6-diphenyl-1,2,4-triazine-3-yl)pyridine; 2,4,6-tri(2-pyridyl)-1,3,5-triazine; 4,4′-dimethyl-2,2′-dipyridyl; 2,2′-biquinoline; di-2-pyridyl glyoxal {2,2′-pyridil }; 2-(2-pyridyl)benzimidazole; 2,2′-bipyrazine; 3-(2-pyridyl )5,6-diphenyl-1,2,4-triazine; 3-(4-phenyl-2-pyridyl)-5-phenyl-1,2,4-triazine; 3-(4-phenyl-2-pyridyl)-5,6-diphenyl-1,2,4-triazine; 2,3,5,6-tetrakis-(2′-pyridyl)-pyrazine; 2,6-pyridinedicarboxylic acid; 2,4,5-trihydroxypyrimidine; phenyl 2-pyridyl ketoxime; 3-amino-5,6-dimethyl-1,2,4-triazine; 6-hydroxy-2-phenyl-3(2H)-pyridazinone; 2,4-pteridinediol {lumazine }; 2,2-′dipyridyl; and 2,3-dihydroxypyridine. Other preferred chelating agents are analogs, homologs and isomers of the above aromatic amines which exhibit at least about 50% inhibition of iron-catalyzed hydroxyl radical formation in the in vitro solution radical assay.

Preferred chelating agents useful in the present invention which fall within the class of carbonyls include the following: di-2-pyridyl ketone; 1,1′-carbonyldiimidazole and 2,2′-pyridil. Other preferred chelating agents are analogs, homologs and isomers of the above carbonyls, which exhibit at least about 50% inhibition of iron-catalyzed hydroxyl radical formation in the in vitro solution radical assay.

Preferred chelating agents useful in the present invention which fall within the class of oximates include the following: 2-furildioxime; 2-furilmonoxime; phenyl 2-pyridyl ketoxime; and 1,2-cyclohexanedione dioxime. Other preferred chelating agents are analogs, homologs and isomers of the above oximates, which exhibit at least about 50% inhibition of iron-catalyzed hydroxyl radical formation in the in vitro solution radical assay.

Preferred chelating agents useful in the present invention which fall within the class of amines include the following: ethylenediamine-N,N-bis-(2-hydroxyphenylacetic acid) dimethyl ester; diethyldithiocarbamic acid; 1-pyrrolidinecarbodithioic acid; and 3-amino-5,6-dimethyl-1,2,4-triazine. Other preferred chelating agents are analogs, homologs and isomers of the above amines, which exhibit at least about 50% inhibition of iron-catalyzed hydroxyl radical formation in the in vitro solution radical assay.

Preferred chelating agents useful in the present invention which fall within the class of carboxylates include the following: 2,3-dihydroxybenzoic acid; 3-hydroxy-5-(hydroxymethyl)-2-methyl-4-pyridine-carboxylic acid {pyridoxic acid}; ethylenediamine-N,N-bis-(2-hydroxyphenylacetic acid) dimethyl ester; and 2,6-pyridinedicarboxylic acid. Other preferred chelating agents are analogs, homologs and isomers of the above carboxylates, which exhibit at least about 50% inhibition of iron-catalyzed hydroxyl radical formation in the in vitro solution radical assay.

Preferred chelating agents useful in the present invention which fall within the class of alkoxides include the following: 5-hydroxy-2-(hydroxymethyl)-4H-pyran-4-one {kojic acid } and pyridoxic acid. Other preferred chelating agents are analogs, homologs and isomers of the above alkoxides, which exhibit at least about 50% inhibition of iron-catalyzed hydroxyl radical formation in the in vitro solution radical assay.

Preferred chelating agents useful in the present invention which fall within the class of enolates include the following: 1,2-dimethyl-3-hydroxypyrid-4-one; 3-hydroxy-2-methyl-4-pyrone; kojic acid; 1 -hydroxy-4-methyl-6-(2,4,4-trimethylpentyl)-2(1H)-pyridone {piroctone olamine-Octopirox); and 6-cyclohexyl-1-hydroxy-4-methyl-2(1H)-pyridinone {Ciclopirox}. Other preferred chelating agents are analogs, homologs and isomers of the above enolates, which exhibit at least about 50% inhibition of iron-catalyzed hydroxyl radical formation in the in vitro solution radical assay.

Preferred chelating agents useful in the present invention which fall within the class of phenoxides include the following: Octopirox; 6-hydroxy-2-phenyl-3(2H)-pyridazinone; Ciclopirox; 2,3-dihydroxybenzoic acid; 4,5-dihydroxy-1,3-benzene-disulfonic acid {Tiron }; ethylenediamine-N,N-bis-(2-hydroxyphenylacetic acid) dimethyl ester; pyridoxic acid; 2,3-dihydroxypyridine; 2,4,5-trihydroxypyrimidine; and 2,3-dihydroxynaphthalene. Other preferred chelating agents are analogs, homologs and isomers of the above phenoxides, which exhibit at least about 50% inhibition of iron-catalyzed hydroxyl radical formation in the in vitro solution radical assay.

Preferred chelating agents useful in the present invention which fall within the class of catecholates include the following: 2,3-dihydroxynaphthalene; 2,4,5-trihydroxypyrimidine; kojic acid; 2,3-dihydroxypyridine; 3-hydroxy-2-methyl-4-pyrone; Tiron; 2,3-dihydroxybenzoic acid; 4-(2-amino-1-hydroxyethyl)-1,2-benzenediol; Ciclopirox; and Octopirox. Other preferred chelating agents are analogs, homologs and isomers of the above catecholates, which exhibit at least about 50% inhibition of iron-catalyzed hydroxyl radical formation in the in vitro solution radical assay.

Preferred chelating agents useful in the present invention which fall within the class of hydroxy acids include the following: 2,3-dihydroxybenzoic acid; and pyridoxic acid. Other preferred chelating agents are analogs, homologs and isomers of the above hydroxy acids which exhibit at least about 50% inhibition of iron-catalyzed hydroxyl radical formation in the in vitro solution radical assay.

Preferred chelating agents useful in the present invention which fall within the class of hydroxamates include the following: N-benzoyl -N-phenyl-hydroxylamine; desferrioxamine B {Desferal}; Ciclopirox; and Octopirox. Other preferred chelating agents are analogs, homologs and isomers of the above hydroxamates, which exhibit at least about 50% inhibition of iron-catalyzed hydroxyl radical formation in the in vitro solution radical assay.

Preferred chelating agents useful in the present invention which fall within the class of ketoenolates include the following: kojic acid. Other preferred chelating agents are analogs, homologs and isomers of the above ketoenolates, which exhibit at least about 50% inhibition of iron-catalyzed hydroxyl radical formation in the in vitro solution radical assay.

Preferred chelating agents useful in the present invention which fall within the class of mercaptides include the following: diethyldithiocarbamic acid; and 1-pyrrolidinecarbodithioic acid. Other preferred chelating agents are analogs, homologs and isomers of the above mercaptides, which exhibit at least about 50% inhibition of iron-catalyzed hydroxyl radical formation in the in vitro solution radical assay.

Preferred chelating agents useful in the present invention which fall within the class of hydroxy aromatic amines include the following: 5,7-dichloro-8-hydroxyquinoline. Other preferred chelating agents are analogs, homologs and isomers of the above hydroxy aromatic amines which exhibit at least about 50% inhibition of iron-catalyzed hydroxyl radical formation in the in vitro solution radical assay.

A preferred chelating agent useful in the present invention which falls within the class of polyphosphates is phytic acid (inositol hexaphosphate).

Preferred chelating agents useful in the present invention which fall within the class of aromatic hydroxy acids include the following: 2,3-dihydroxybenzoic acid; pyridoxic acid; and 2,6-pyridinedicarboxylic acid. Other preferred chelating agents are analogs, homologs and isomers of the above aromatic hydroxy acids which exhibit at least about 50% inhibition of iron-catalyzed hydroxyl radical formation in the in vitro solution radical assay.

More preferred chelating agents for use in the compositions and methods of the present invention include the following: 2,2′-dipyridylamine; o-phenanthroline; di-2-pyridyl ketone; 2-furildioxime; 2-furilmonoxime; 2,3-bis(2-pyridyl) pyrazine; Octopirox; 2,3-dihydroxybenzoic acid; ethylenediamine-N,N-bis-(2-hydroxyphenylacetic acid), dimethyl ester; 1,1′-carbonyldiimidazole; 1,2-dimethyl-3-hydroxypyrid-4-one; 2,4,6-tri(2-pyridyl)-1,3,5-triazine; 1-pyrrolidinecarbodithioic acid; diethyldithiocarbamic acid; Ciclopirox; 2,2′-dipyridyl; 1,2-cyclohexanedione dioxime; 3-hydroxy-2-methyl-4-pyrone; 2,3-bis(2-pyridyl)-5,6-dihydropyrazine; 3-(4-phenyl-2-pyridyl)-5-phenyl-1,2,4-triazine; kojic acid; 2,3-dihydroxypyridine; 2,2′-biquinoline; 2,2′-bipyrazine; 3-(2-pyridyl)-5,6-diphenyl-1,2,4-triazine; 4-(2-amino-1-hydroxyethyl)-1,2-benzenediol; and 4,4′-dimethyl-2,2′-dipyridyl.

Still more preferred chelating agents for use in the compositions and methods of the present invention including the following: 2,2′-dipyridylamine; o-phenanthroline, di-2-pyridyl ketone; 2-furildioxime; 2-furilmonoxime; 2,3-bis(2-pyridyl) pyrazine; Octopirox; 2,3-dihydroxybenzoic acid; ethylenediamine-N,N-bis-(2-hydroxyphenylacetic acid), dimethyl ester; 1,1′-carbonyldiimidazole; 1,2-dimethyl-3-hydroxypyrid-4-one; 2,4,6-tri(2-pyridyl)-1,3,5-triazine; 1-pyrrolidinecarbodithioic acid; diethyldithiocarbamic acid; and Ciclopirox.

More preferred still chelating agents for use in the compositions and methods of the present invention including the following: 2,2′-dipyridylamine; o-phenanthroline, di-2-pyridyl ketone; 2-furildioxime; 2-furilmonoxime; 2,3-bis(2-pyridyl) pyrazine; Octopirox; 2,3-dihydroxybenzoic acid; and ethylenediamine-N,N-bis-(2-hydroxyphenylacetic acid), dimethyl ester.

Preferred chelating agents for use in the compositions and methods of the present invention include the following: 2,2′-dipyridylamine; o-phenanthroline; di-2-pyridyl ketone; 2-furildioxime and 2-furilmonoxime, with the most preferred being oximes.

The preferred chelating agents listed hereinabove are generally commercially available from one or more of the following suppliers: Aldrich Chemical Company, Milwaukee, Wis.; G.F.S. Chemicals, Columbus, Ohio; Dojindo Laboratories, Kumamoto, Japan; Sigma Chemical Company, St. Louis, Mo.; Ciba-Geigy, Summit, N.J.; Strem Chemicals, Newburyport, Mass.; and American Hoechst Corp., Summerville, N.J. A method for synthesizing 1,2-dimethyl-3-hydroxypyrid-4-one is disclosed in Kontoghiorghes, G. J., “L1-1,2-dimethyl-3-hydroxypyrid-4-one”, Drugs of the Future, Vol. 13, No. 5 (1988), pp. 413-415, which is hereby incorporated by reference.

A safe and protectively effective amount of a chelating agent is used in the compositions of the present invention. Typically, this is from about 0.01% to about 10%, preferably from about 1 to about 5%, most preferably from about 2% to about 5%, of the composition.

It is important to note that the chelating agent works in the skin to prevent damaging reactions in the skin. Rub-off, wear-off or wash-off of the active ingredient, are essentially irrelevant with the present invention because the chelating agent penetrates the skin to work. Furthermore, it is not necessary to keep an even coating of the active of the present invention on the skin for the entire exposure period. The chelating agent can be applied to the skin up to four hours or longer prior to radiation exposure.

Carriers

In addition to the active agent, the compositions of the present invention contain a safe and effective amount of an acceptable carrier. The term “acceptable topical carrier” encompasses both pharmaceutically-acceptable carriers and cosmetically-acceptable carriers, and it encompasses substantially nonirritating compatible components (either taken alone or in mixtures) which are suitable for delivering the active component to the skin. The term “compatible”, as used herein, means that the components of the carrier must be capable of being commingled with the chelating agent, and with each other, in a manner such that there is no interaction which would substantially reduce the efficacy of the composition during use for protecting the skin from the effects of high-energy radiation. These carriers must, of course, be of sufficiently high purity and sufficiently low toxicity to render them suitable for chronic topical administration to the skin of humans or lower animals. The term “safe and effective amount” of carrier means an amount sufficient to deliver the chelating agent to the skin but not so much as to cause any side effects or skin reactions, generally from about 50% to about 99%, preferably from about 90% to about 98%, of the composition.

Variations in formulation of these carriers will result in a wide variety of products which fall within the scope of the present invention. These product types can be divided into two classes: pharmaceutical/cosmetic compositions and cleaning compositions.

Pharmaceutical/ Compositions

The pharmaceutical/cosmetic compositions of the present invention may be made into a wide variety of product types. These include, for example, lotions, creams, oils, gels, sticks, sprays, ointments and pastes. These product types may comprise either of two basic types of carrier systems, i.e., solutions and emulsions.

The pharmaceutical/ compositions of the present invention formulated as solutions typically include a pharmaceutically-acceptable organic solvent. The term, “pharmaceutically-acceptable organic solvent”, refers to an organic solvent, which, in addition to being capable of having dispersed or dissolved therein the chelating agent, also possesses acceptable safety (e.g. irritation and sensitization characteristics), as well as good aesthetic properties (e.g., does not feel greasy or tacky). The most typical example of such a solvent is water. Examples of other suitable organic solvents include: propylene glycol, polyethylene glycol (200-600), polypropylene glycol (425-2025), glycerol, 1,2,4-butanetriol, sorbitol esters, 1,2,6-hexanetriol, ethanol, isopropanol, butanediol, and mixtures thereof. These solutions contain from about 1% to about 20%, preferably from about 2% to about 10%, of the chelating agent, and from about 80% to about 99%, preferably from about 90% to about 98%, of an acceptable organic solvent.

If the pharmaceutical compositions of the present invention are formulated as an aerosol and applied to the skin as a spray-on, a propellant is added to a solution composition. Examples of propellants useful herein include the chlorinated, fluorinated, chloro-fluorinated lower molecular weight hydrocarbons and non-chloro-fluorinated hydrocarbon propellants. Other propellants useful in the present invention include lower molecular weight hydrocarbon mixtures (e.g., the mixture of butane, isobutane and propane known commercially as Propellant A46, made by Phillips Chemical Co., a subsidiary of Phillips Petroleum Company), ethers and halohydrocarbons such as dimethyl ether or dichlorodifluoromethane alone or mixtures thereof with dichlorotetrafluoroethane. Mixtures of hydrocarbon and halohydrocarbon propellants and nitrous oxide may also be used. Nitrogen and carbon dioxide can also be used as propellant gases. They are used at a level sufficient to expel the contents of the container. Examples of non-chloro-fluorinated hydrocarbon propellants are: HCFC 123, HCFC 124, HCFC141b, HCFC 225, HCFC 125, FC-C 51-12, DYMEL A, DYMEL 152a, HFC 134a and HFC 227ea. A more complete disclosure of propellants useful herein can be found in Sagarin, Cosmetics Science and Technology, 2nd Edition, Vol. 2, pp. 443-465 (1972), incorporated herein by reference.

Alternatively, emollients may comprise the carrier system of the present invention formulated as a solution. Such compositions contain from about 1% to about 20% of the chelating agent and from about 2% to about 50% of a pharmaceutically-acceptable emollient.

As used herein, “emollients” refer to materials used for the prevention or relief of dryness, as well as for the protection of the skin. A wide variety of suitable emollients are known and may be used herein. Sagatin, Cosmetics, Science and Technology, 2nd Edition, Vol. 1, pp. 32-43 (1972), incorporated herein by reference, contains numerous examples of suitable materials.

A lotion can be made from a solution carrier system. Lotions typically comprise from about 1% to about 20%, preferably from about 2% to about 10%, of the chelating agent; from about 1% to about 20%, preferably from about 5% to about 10%, of an emollient; and from about 50% to about 90%, preferably from about 60% to about 80%, water. Another type of product that may be formulated from a solution carrier system is a cream. A cream of the present invention would comprise from about 1% to about 20%, preferably from about 2% to about 10%, of the chelating agent; from about 5% to about 50%, preferably from about 10% to about 20%, of an emollient, and from about 45% to about 85%, preferably from about 50% to about 75%, water.

Yet another type of product that may be formulated from a solution carrier system is an ointment. An ointment may comprise a simple base of animal or vegetable oils or semi-solid hydrocarbons (oleaginous). Ointments may also comprise absorption ointment bases which absorb water to form emulsions. Examples of such ointment bases include, anhydrous lanolin and hydrophilic petrolatum. Emulsion ointment bases may be oil-in-water or water-in-oil emulsions. Ointment carriers may also be water-soluble. Examples of such ointment carriers include glycol ethers, propylene glycols, polyoxyl stearates, and polysorbates. An ointment may also comprise from about 2% to about 10% of an emollient plus from about 0.1% to about 2% of a thickening agent. Examples of suitable thickening agents include: cellulose derivatives (e.g., methyl cellulose and hydroxy propylmethyl cellulose), synthetic high molecular weight polymers (e.g., carboxyvinyl polymer and polyvinyl alcohol), regiment hydrocolloids (e.g., karaya gum and tragacanth gum), clay thickeners (e.g., colloidal magnesium aluminum silicate and bentonite), and carboxyvinyl polymers (Carbopols.RTM.—by B. F. Goodrich Company, such polymers are described in detail in U.S. Pat. No. 2,798,053, Brown, issued Jul. 2, 1975, incorporated herein by reference). A more complete disclosure of thickening agents useful herein can be found in Segatin, Cosmetics, Science and Technology, 2nd Edition, Vol. 1, pp. 72-73 (1972), incorporated herein by reference.

If the carrier is formulated as an emulsion, from about 1% to about 10%, preferably from about 2% to about 5%, of the carrier system comprises an emulsifier. Emulsifiers may be nonionic, anionic or cationic. Suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and McCutcheon's Detergents and Emulsifiers, North American Edition, pages 317-324 (1986); the disclosures of which are incorporated herein by reference. Preferred emulsifiers are anionic or nonionic, although the other types may also be used.

Single emulsion skin care preparations, such as lotions and creams, of the oil-in-water type and water-in-oil type are well-known in the cosmetic art and are also useful in the present invention. Multiphase emulsion compositions, such as the water-in-oil-in-water type, as disclosed in U.S. Pat. No. 4,254,105, Fakuda et al., issued Mar. 3, 1981, herein incorporated by reference, are also useful in the present invention. In general, such single or multiphase emulsions contain water, emollients and emulsifiers as essential ingredients.

Triple emulsion carrier systems comprising an oil-in-water-in-silicone fluid emulsion composition as disclosed in U.S. patent application Ser. No. 022,876, Figueroa, et al., filed Mar. 6, 1987, herein incorporated by reference, are also useful in the present invention. This triple emulsion carrier system can be combined with from about 1% to about 20%, preferably from about 2% to about 10%, of the chelating agent to yield the pharmaceutical composition of the present invention.

Another emulsion carrier system useful in the pharmaceutical/cosmetic compositions of the present invention is a microemulsion carrier system. Such a system comprises from about 9% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other nonionics; and from about 7% to about 20% water. This carrier system is combined with from about 2% to about 10% of the chelating agent.

Lotions and creams can be formulated as emulsions as well as solutions. Typically such lotions comprise from about 1% to about 20%, preferably from about 2% to about 10%, of the chelating agent; from about 1% to about 20%, preferably from about 5% to about 10%, of an emollient; from about 25% to about 75%, preferably from about 45% to about 95%, water; and from about 1% to about 10%, preferably from about 2% to about 5%, of an emulsifier. Such creams would typically comprise from about 1% to about 20%, preferably from about 2% to about 10%, of the chelating agent; from about 1% to about 20%, preferably from about 5% to about 10%, of an emollient; from about 20% to about 80%, preferably from about 30% to about 70%, water; and from about 1% to about 10%, preferably from about 2% to about 5%, of an emulsifier.

If the pharmaceutical compositions of the present invention are formulated as gels a suitable amount of a thickening agent, as disclosed supra, is added to a cream or lotion formulation.

The topical pharmaceutical compositions of the present invention may contain, in addition to the aforementioned components, a wide variety of additional oil-soluble materials and/or water-soluble materials conventionally used in topical compositions, at their art-established levels.

Among the optional oil-soluble materials are nonvolatile silicone fluids, such as polydimethyl siloxanes with viscosities ranging from about 10 to about 100,000 centistokes at 25° C. These siloxanes are useful to enhance skin feel and are available from Dow Corning Corporation as the Dow Corning 200 series. These optional oil-soluble materials may comprise up to about 20% of the total composition, preferably up to about 10%.

Various water-soluble materials may also be present in the compositions of this invention. These include humectants, such as glycerol, sorbitol, propylene glycol, polyethylene glycol, alkoxylated glucose, hexanetriol, ethyl cellulose, polyvinyl alcohol, carboxymethyl cellulose, vegetable gums and clays such as Veegum.RTM. (magnesium aluminum silicate, R. T. Vanderbilt, Inc.); proteins and polypeptides; preservatives such as the methyl, ethyl, propyl and butyl esters of hydroxybenzoic acid (Parabens-Mallinckrodt Chemical Corporation), EDTA, methylisothiazolinone and imidazolidinyl ureas (Germall 115-Sutton Laboratories); and an alkaline agent such as sodium hydroxide or potassium hydroxide to neutralize, if desired, part of the fatty acids or thickener which may be present. In addition, the topical compositions herein can contain conventional cosmetic adjuvants, such as dyes, opacifiers (e.g., titanium dioxide), pigments and perfumes.

The pharmaceutical/cosmetic compositions of the present invention may also include a safe and effective amount of a penetration-enhancing agent. By “safe and effective amount” is meant an amount sufficient to enhance penetration of the chelating agent into the skin but not so much as to cause any side effects or skin reactions, generally from about 1% to about 5% of the composition. Examples of useful penetration enhancers, among others, are disclosed in U.S. Pat. No. 4,537,776, Cooper, issued Aug. 27, 1985; U.S. Pat. No. 4,552,872, Cooper et al., issued Nov. 12, 1985; U.S. Pat. No. 4,557,934, Cooper, issued Dec. 10, 1985; U.S. Pat. No. 4,130,667, Smith, issued Dec. 19, 1978; U.S. Pat. No. 3,989,816, Rhaadhyaksha, issued Nov. 2, 1976; U.S. Pat. No. 4,017,641, DiGiulio, issued Apr. 12, 1977; and European Patent Application 0043738, Cooper et al., published Jan. 13, 1982. U.S. Pat. No. 4,537,776 teaches a penetration-enhancing vehicle consisting essentially of a) N-(2-hydroxyethyl) pyrrolidone and b) a cell envelope disordering compound selected from methyl laurate, oleic acid, oleyl alcohol, monoolein, myristyl alcohol, and mixtures thereof, wherein component (a) and (b) are present in a ratio of (a):(b) of about 1:5 to about 500:1 by weight. U.S. Pat. No. 4,557,934 teaches a pharmaceutical composition comprising the penetration enhancing agent 1-dodecylazacycloheptan-2-one, and a penetration enhancing diol or cycloketo compound selected from the group consisting of: 1,2-propanediol, 1,3-propanediol, 1,2-butanediol, pyrrolidone; 1-(2-hydroxyethyl)-azacyclopentan-2-one, and mixtures thereof. U.S. Pat. No. 4,130,667 describes a penetration enhancer comprising:
(a) at least about 0.1% by weight of a sugar ester selected from sucrose monooctanoate, sucrose monodecanoate, sucrose monolaurate, sucrose monomyristate, sucrose monopalmitate, sucrose monostearate, sucrose monooleate, and sucrose dioleate; and
(b) at least about 0.1% by weight of a phosphine oxide compound selected from octyldimethyl phosphine oxide, nonyl dimethyl phosphine oxide, decyl dimethyl phosphine oxide, undecyl dimethyl phosphine oxide, dodecyl dimethyl phosphine oxide, 2-hydroxydecyl dimethyl phosphine oxide, 2-hydroxy undecyl dimethyl phosphine oxide, and 2-hydroxy dodecyl dimethyl phosphine oxide. Sulfoxides may be used in some executions in place of the phosphine oxide.

Other conventional skin care product additives may also be included in the compositions of the present invention, if desired. For example, collagen, hyaluronic acid, elastin, hydrolysates, primrose oil, jojoba oil, epidermal growth factor, soybean saponins, mucopolysaccharides, and mixtures thereof may be used.

Various vitamins may also be included in the compositions of the present invention. For example, Vitamin A, and derivatives thereof, Vitamin B.sub.2, biotin, pantothenic, Vitamin D, and mixtures thereof may be used.

Combination Actives

An agent may also be added to any of the compositions of the present invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157, Brock, issued May 5, 1987, which is incorporated herein by reference. The disclosed skin substantivity agent comprises the polymeric form of two monomers, ethylene and acrylic acid, to yield the following: (CH2—CH2)ₓ(CH₂—CH₂)_{y}COOH, wherein the ratio of x:y is from about 1:24 to about 1:9, and wherein the weight average molecular weight of the molecule is from about 3500 to about 4500, preferably from about 4000 to about 4300. These copolymers are preferably included in an oil-in-water emulsion composition comprising: a) from about 1% to about 20% of the chelating agent; b) from about 0.25% to about 3% of the ethylene-acrylic acid copolymer as described above; c) from about 2% to about 10% of an emulsifier; and d) from about 70% to about 90% of water, wherein the ratio of protective agents to the copolymer is from about 12:1 to about 15: 1.

For oral administration or administration by injection, the carriers may comprise any of those commonly employed in the art provided that they are pharmaceutically acceptable and compatible with the active components of the composition to be administered.

In addition, the carrier may also impart improved properties both to the composition and to the active, for example, increased bioavailability and sustained efficacy (by the stabilization of the active against hydrolysis or other attack which may occur during shelf life or use and the like), and other similar synergystic benefits. Thus it is recognized that certain actives may optionally be stabilized in formulation as well as by their design. Examples of such formulations include buffered compositions, emulsions, encapsulation (and microencapsulation, such as described in “Microcapsules-Innovative, Versatile Product Delivery: Batelle Technical Inputs to Planning Report #33 Columbus, Ohio (1983)) nonaqueous compositions, anhydrous compositions and the like.

Some examples of substances which can serve as pharmaceutically-acceptable carriers are sugars such as lactose, glucose and sucrose; starches such as corn starch and potato starch; cellulose and its derivatives, such as sodium carboxymethylcellulose, ethylcellulose, cellulose acetate; beta-cyclodextrin (.beta.-cyclodextrin); powdered tragacanth; malt; gelatin; talc; stearic acid; magnesium stearate; calcium sulfate; vegetable oils such as peanut oil, cottonseed oil, sesame oil, olive oil, corn oil and oil of theobroma; polyols such as propylene glycol, glycerine, sorbitol, mannitol, and polyethylene glycol; sugar; alginic acid; pyrogen-free water; isotonic saline; buffer solutions; cocoa butter (suppository base); emulsifiers, such as the Tweens.RTM.; as well as other non-toxic compatible substances used in pharmaceutical formulations. Wetting agents and lubricants such as sodium lauryl sulfate, as well as coloring agents, flavoring agents, excipients, tableting agents, stabilizers, antioxidants, and preservatives, can also be present. If the compound is to be injected, the injectable carrier depends upon the solubility and stability of the particular compound. Suitable pharmaceutically-acceptable carriers for topical application include those suited for use in creams, gels, tapes and the like; and for oral administration include those suited for tablets and capsules.

Pharmaceutically-acceptable carriers suitable for the preparation of unit dosage forms for oral administration and injection are well-known in the art. Their selection will depend on considerations like taste, cost, and/or shelf stability, etc., which are not critical for the purposes of this invention, and can be made without difficulty by a person skilled in the art. Pharmaceutically-acceptable carriers useful in the compositions of this invention are described more fully hereinafter.

A. Oral Dose Forms

Various oral dosage forms can be used, including such solid forms as tablets, capsules, granules, bulk powders and microcapsules of the drug. These oral forms comprise a safe and effective amount of a compound of this invention. Tablets can be compressed, enteric-coated, sugar-coated or film-coated containing suitable binders, lubricants, surfactants, diluents, disintegrating agents, coloring agents, flavoring agents, preservatives, flow-inducing agents, and melting agents. Liquid oral dosage forms include aqueous and nonaqueous solutions, emulsions, suspensions, solutions and/or suspensions reconstituted from non-effervescent granules, containing suitable solvents, preservatives, emulsifying agents, suspending agents, diluents, sweeteners, melting agents, coloring agents, and flavoring agents. Preferred carriers for oral administration include gelatin and propylene glycol. Specific examples of pharmaceutically-acceptable carriers and excipients that may be used in formulating oral dosage forms containing compounds of this invention are described in U.S. Pat. No. 3,903,297, Robert, issued Sep. 2, 1975, incorporated by reference herein. Techniques and compositions for making solid oral dosage forms are described in Marshall, “Solid Oral Dosage Forms,” Modern Pharmaceutics, Vol. 7, (Banker and Rhodes, editors), 359-427 (1979), incorporated herein by reference. Techniques and compositions for making tablets (compressed, formulas and molded), capsules (hard and soft gelatin) and pills are described in Remington's Pharmaceutical Sciences (Arthur Osol, editor), 1553-1593 (1980), incorporated herein by reference.

B. Injectable Dose Forms

The compounds of this invention are also useful when injected. Methods and materials for manufacturing injectables can be found in Remington's Pharmaceutical Sciences, 17ed., 1985, Chapter 85, p. 1518, the disclosures of which are incorporated herein by reference in their entirety.

Anti-Inflammatory Agents

In a preferred composition of the present invention, an anti-inflammatory agent is included as an active along with the chelating agent. The inclusion of an anti-inflammatory agent enhances the protection benefits of the compositions. It has now been discovered that the chronic use of anti-inflammatories also greatly reduces radio-aging of the skin resulting from chronic exposure to high-energy radiation. It has also been discovered that the combination of an anti-inflammatory agent and the chelating agent provides greater protection than is provided by each active alone. Furthermore, the combination provides greater protection than is provided by the sum of the effects of each active alone.

A safe and protectively effective amount of an anti-inflammatory agent may be added to the compositions of the present invention. By “safe and protectively effective” amount is meant an amount sufficient to provide protection when the composition is properly applied, but not so much as to cause any side effects or adverse skin reactions; generally from about 0.1% to about 10%, preferably from about 0.5% to about 5%, of the composition. The exact amount of anti-inflammatory agent to be used in the compositions will depend on the particular anti-inflammatory agent utilized since such agents vary widely in potency. Steroidal anti-inflammatory agents, including but not limited to, corticosteroids such as hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use in the present invention is hydrocortisone.

A second class of anti-inflammatory agents which is useful in the compositions of the present invention includes the nonsteroidal anti-inflammatory agents. The variety of compounds encompassed by this group are well-known to those skilled in the art. For detailed disclosure of the chemical structure, synthesis, side effects, etc., of non-steroidal anti-inflammatory agents, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974), incorporated herein by reference.

Specific non-steroidal anti-inflammatory agents useful in the composition of the present invention include, but are not limited to:

1) the oxicams, such as piroxicam, isoxicam, tenoxicam, sudoxicam, and CP-14,304; 2) the salicylates, such as aspirin, disalcid, benorylate, trilisate, safapryn, solprin, diflunisal, and fendosal;

3) the acetic acid derivatives, such as diclofenac, fenclofenac, indomethacin, sul indac, tolmetin, isoxepac, furofenac, tiopinac, zidometacin, acematacin, fentiazac, zomepiract, clidanac, oxepinac, and felbinac;

4) the fenamates, such as mefenamic, meclofenamic, flufenamic, niflumic, and tolfenamic acids;

5) the propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and

6) the pyrazoles, such as phenybutazone, oxyphenbutazone, feprazone, azapropazone, and trimethazone.

Mixtures of these non-steroidal anti-inflammatory agents may also be employed, as well as the pharmaceutically-acceptable salts and esters of these agents. For example, etofenamate, a flufenamic acid derivative, is particularly useful for topical application. Of the nonsteroidal anti-inflammatory agents, ibuprofen, naproxen, flufenamic acid, mefenamic acid, meclofenamic acid, piroxicam and felbinac are preferred; ibuprofen, naproxen, and flufenamic acid are most preferred.

Another class of anti-inflammatory agents which are useful in the present invention are the anti-inflammatory agents disclosed in U.S. patent application Ser. No. 879,863, Loomans et al., filed Jun. 27, 1986. This application discloses a class of non-steroidal anti-inflammatory compounds which comprise specifically substituted phenyl compounds, especially substituted 2,6-di-tert-butyl phenol derivatives. For example, compounds selected from 4-(4′-pentyn-3′-one)-2,6-di-t-butylphenol; 4-(5′-hexynoyl)-2,6-di-t-butylphenol; 4-((S)-(−)-3′-methyl-5′-hexynoyl)-2,6-di-t-butylphenol; 4-((R)-(+)-3′-methyl-5′-hexynoyl)-2,6-di-t-butylphenol; and 4-(3′,3′-dimethoxypropionyl)-2,6-di-t-butylphenol are useful in the present invention.

Yet another class of anti-inflammatory agents which are useful in the present invention are those disclosed in U.S. patent application Ser. No. 051,446, Mueller, filed May 18, 1987, the entire contents and disclosure of which is incorporated herein by reference. This application discloses compounds and diastereomeric mixtures of specific 2-naphthyl-containing ester compounds, especially naproxen ester and naproxol ester compounds, having two or more chiral centers. For example, compounds selected from (S)-naproxen- (S)-2-butyl ester, (S)-naproxen-(R)-2-butylester, (S)-naproxol-(R)-2-methyl butyrate, (S)-naproxol-(S)-2-methyl butyrate, diasteromeric mixtures of (S)-naproxen-(S)-2-butyl ester and (S)-naproxen-(R)-2-butyl ester, and diasteromeric mixtures of (S)-naproxol-(R)-2-methyl butyrate and (S)-naproxol-(S)-2-methyl butyrate are useful in the present invention.

Finally, so-called “natural” anti-inflammatory agents are useful in the present invention. For example, phyto sterol (e.g., sterols from canola oil or soy oil), willow bark extract, candelilla wax, alpha bisabolol, aloe vera, Manjistha (extracted from regiments in the genus Rubia, particularly Rubia Cordifolia), and Guggal (extracted from regiments in the genus Commiphora, particularly Commiphora Mukul), may be used.

An even more preferred composition of the present invention comprises a chelating agent and an anti-inflammatory agent together for protection. Such a composition comprises from about 0.01% to about 10%, preferably from about 2% to about 5%, of the chelating agent; and from about 0.1% to about 5%, preferably from about 0.5% to about 2%, of an anti-inflammatory agent.

The protection compositions of the present invention may comprise, in addition to the chelating agent, a safe and protectively effective amount of a radical scavenging compound. By “safe and protectively effective amount” is meant an amount sufficient to provide protection when the composition is properly applied, but not so much as to cause any side effects or adverse skin reactions; generally from about 0.1% to about 20%, preferably from about 1% to about 5%, of the composition. Examples of such radical scavenging compounds are ascorbic acid (Vitamin C) and its salts and derivatives (e.g., magnesium ascorbyl phosphate, sodium ascorbyl phosphate. Ascorbyl palmitate, etc.), tocopherol (Vitamin E), tocopherol esters (e.g., tocopheryl acetate, tocopheryl succinate, tocopheryl sorbate, butylated hydroxy benzoic acids and their salts, 6-hydroxy-2,5,7,8-tetramethylchroman- 2-carboxylic acid (commercially available under the tradename Trolox.RTM.), gallic acid and its alkyl esters (especially propyl gallate), uric acid and its salts and alkyl esters, sorbic acid and its salts, the ascorbyl esters of fatty acids, amines (e.g., N,N-diethylhydroxylamine, aminoguanidine), sulfhydryl compounds (e.g., glutathione), and dihydroxyfumaric acid and its salts. Additionally, catechins and polyphenols (e.g., those found in green tea extract) and flavonoids (e.g., isoflavones such as genistein, and daidxein which are found in soy extracts, flavones, chalcones, flavanones, coumarins, etc.) can be used. Each of these compounds has radioprotecting capabilities. However, the use of the radical scavenger tocopheryl esters (e.g., tocopherol sorbate) in the present invention in combination with the chelating agent is preferred.

From about 0.1% to about 5% of these radical scavenging compounds may be used in the present invention in combination with the levels of chelating agent taught herein. Exact amounts will vary depending on which particular compound is used as these compounds vary somewhat in potency.

The present invention further relates to a method for protecting the skin of humans and lower animals from the deleterious effects of high-energy radiation.

Such a method comprises applying to the skin of the human or lower animal a safe and protectively effective amount of the specifically defined chelating agents, which meet the criteria, described supra. This may be accomplished by using a composition comprising of the chelating agent as described in the present application. The term “safe and protectively effective amount”, as used here (and hereinafter regarding other types of agents), means an amount of agent sufficient to substantially reduce the deleterious effects of high-energy radiation to skin but not so much as to cause serious side effects or adverse skin reactions. Typically a safe and protectively effective amount is from about 0.001 mg to about 1.0 mg, preferably from about 0.01 mg to about 0.5 mg, more preferably from about 0.05 mg to about 0.2 mg of the chelating agent per cm² skin. The chelating agent may be simply spread or sprayed onto the skin or may preferably be rubbed into the skin to enhance penetration. The chelating agent is applied in conjunction with high-energy radiation exposure. The chelating agent works best if applied prior to or concomitantly with the radiation exposure. In addition, because of the mechanism by which the chelators appear to work (i.e., the chelation of metal ion formed in the skin), the chelating agents may also provide benefits if applied after exposure. Such application may take place immediately after and up to several hours after exposure (e.g., nightly application of a skin moisturizing product), but preferably takes place within about 30 minutes after exposure. The application of the chelating agent may be done several days to immediately prior to exposure. This is because the active agent penetrates the skin to work and thus is not as susceptible to rub-off, wash-off or wear-off. For protection against chronic damage, application of the chelating agent several times daily; generally from about 2 times to about 5 times, preferably 2 times daily is preferred.

The chelating agent may be simply spread over the skin, or rubbed into the skin to enhance penetration of the chelating agent. The actives are applied in conjunction with high-energy radiation exposure, i.e., prior to, during, or after exposure. For protection against acute damage from radiation, application of the actives just prior to exposure may be sufficient. For protection against chronic damage from radiation, application 2 to 5 times daily, preferably, about 2 times daily, is preferred.

The combination of chelating agent plus anti-inflammatory agent and/or free radical scavenging agent (e.g., antioxidant) may be applied prior to, concomitantly with, or after high-energy radiation exposure. More specifically, the combination may be applied up to about several days prior to exposure, up to several hours after exposure, or any time in between. For protection against acute damage from high-energy radiation, application of the chelating agent and the anti-inflammatory agent just prior to exposure, or immediately following exposure, may be sufficient. For protection against chronic damage, application of the chelating agent, the anti-inflammatory agent and/or free radical scavenging agent several times daily, e.g., from about times to about 5 times, preferably about 2 times daily, is preferred.

The following examples further describe and demonstrate the preferred embodiments within the scope of the present invention. The examples are given solely for the purpose of illustration, and are not to be construed as limitations of the present invention since many variations thereof are possible without departing from its spirit and scope.

All percentages and ratios herein are by weight, unless otherwise specified.

### Topical Dose Forms

### EXAMPLE I

A topical composition is prepared by combining the following components utilizing conventional mixing techniques.

| Component | % Weight |
|---|---|
| Ethanol:Propylene Glycol:Water (1:1:2) | 95.0 |
| Di-(2-Furyl) Ethanedione Amphi-Dioxime | 5.0 |

In a suitable vessel, the Di-(2-Furyl) Ethanedione Amphi-Dioxime is dissolved in the ethanol:propylene glycol:water with stirring. Use of an amount of the composition to deposit about 0.02 mg of the active agent per cm² of skin is appropriate. The composition is applied to the skin site to be exposed to radiation. The composition is applied three times daily for two days prior to radiation exposure.

### EXAMPLE II

A topical composition is prepared by combining the following components utilizing conventional mixing techniques.

| Component | % Weight |
|---|---|
| Ethanol | 95.0 |
| Di-(2-Furyl) Ethanedione Amphi-Dioxime | 5.0 |

In a suitable vessel, the Di-(2-Furyl) Ethanedione Amphi-Dioxime is dissolved in the ethanol with stirring. Use of an amount of the composition to deposit about 0.02 mg of the active agent per cm² of skin is appropriate. The composition is applied to the skin site to be exposed to radiation. The composition is applied three times under occluded patch at 24, 12 and 2 hours prior to radiation exposure.

### EXAMPLE III

A topical composition is prepared by combining the following components utilizing conventional mixing techniques.

| Component | % Weight |
|---|---|
| Ethanol | 49.00 |
| Propylene glycol | 25.00 |
| Deionized water | 25.00 |
| Octopirox | 1.00 |

In a suitable vessel, the octopirox is dissolved in the ethanol with stirring. Propylene glycol and deionized water are added with stirring. The composition is applied to the skin site to be exposed to radiation. An amount of the composition is applied to deposit about 0.05 mg of the active agent per cm² of skin. The composition is applied 2 hours prior to and immediately after radiation exposure.

### EXAMPLE IV

A nonionic oil-in-water emulsion is prepared by combining the following components utilizing conventional mixing techniques:

| Component | % Weight |
|---|---|
| Deionized Water | 79.33 |
| Propylene Glycol | 3.00 |
| Cetyl Alcohol | 2.50 |
| Stearyl Alcohol | 2.50 |
| Laureth 23 | 2.00 |
| C₁₂₋₁₅ Alcohols Benzoate | 2.00 |
| EDTA | 0.37 |
| Methylparaben | 0.20 |
| Propylparaben | 0.10 |
| Desferrioxamine | 8.0 |

The composition is applied to the skin site to be exposed to radiation. Use of an amount of the composition sufficient to deposit about 0.004 mg per cm² skin of the active agent is appropriate. The composition is applied twice a day for 5 days before and 5 days after radiation exposure.

### Oral Dose Forms

### EXAMPLE V

A composition for oral administration is prepared by combining the following Component:

| | | |
|---|---|---|
| 2-furildioxime (FDO) | 1 | kg |
| Sesame oil | to 4 | liters |

The FDO is suspended in the sesame oil with the aid of sonication and is packaged in soft gelatin capsules using methods known in the art. Two of the resulting capsules, each containing 250 mg of the active, are administered to a 60 kg human for 1 week prior to and 1 week following radiation exposure.

### EXAMPLE VI

A composition for oral administration is prepared by combining the following:

Component

| | | |
|---|---|---|
| 2-furilmonoxime (FMO) | 250 | g |
| Propylene glycol | 1800 | ml |
| Ethyl alcohol | 175 | ml |
| Distilled water | 75 | ml |
| Artificial Cherry flavor | 10 | ml |
| FD&C Red #40 | 0.2 | g |

The above ingredients are combined to produce a syrup and are packaged under sterile conditions in 6 oz. bottles. One teaspoon of this formulation is administered to a 70 kg human, twice daily each day for 7 days prior and 7 days after radiation exposure.

### EXAMPLE VII

Tablets are prepared by conventional methods, such as mixing and direct compaction, formulated as follows:

| Component | mg per tablet |
|---|---|
| piroctone | 500 |
| Microcrystalline cellulose | 400 |
| Sodium Starch glycolate | 60 |
| Magnesium stearates | 10 |

One tablet is taken orally twice daily week prior and 1 week after radiation exposure.

### Intravenous Dose Form

### EXAMPLE VIII

A composition for subcutaneous injection is prepared by combining the following ingredients using conventional methods.

| Component | % Weight |
|---|---|
| 0.9% Normal Saline, inj. USP | 99.5 |
| Phytic Acid | 0.5 |
| NaOH | pH adjust to 5.5 |

The above ingredients are combined to prepare an injectable dose form. Three hours prior to radiation exposure, 20 ml of the solution are continuously infused over a 3-hour period at the skin site to be exposed to radiation.

What is claimed is:

1. A method of inhibiting the deleterious effects on mammalian skin tissue by radiation treatment therapy comprising administering to a patient in need thereof, in conjunction with exposure of the skin tissue to radiation, a safe and effective amount of a chelating agent.

2. The method of claim 1 wherein said chelating agent is applied topically to said patient.

3. The method of claim 1 wherein said chelating agent is administered orally or intravenously to said patient.

4. The method of claim 1 wherein the chelating agent is an aromatic amine, carbonyl compound, oximate, amine, carboxylate, alkoxide, enolate, phenoxide, catecholate, hydroxy acid, hydroxamate, ketoenolate, mercaptide, hydroxy aromatic amine, aromatic hydroxy acid, polyphosphate or a mixture thereof.

5. The method of claim 1 wherein the chelating agent is 2,2′-dipyridylamine; 1,10-phenanthroline; di-2-pyridylketone; 2-furildioxime; 2-furilmonoxime; 2,3-bis(2-pyridyl)pyrazine; 1-hydroxy-4-methyl-6-(2,4,4-trimethylpentyl)-2(1H)-pyridone; 2,3-dihydroxybenzoic acid; ethylenediamine-N,N-bis(2-hydroxyphenylacetic acid), dimethyl ester; 1,1′-carbonyldiimidazole; 1,2-dimethyl-3-hydroxypyrid-4-one; 2,4,6-tri(2-pyridyl)-1,3,5-triazine; 1-pyrrolidinecarbodithioic acid; diethyldithiocarbamic acid; 6-cyclohexyl-1-hydroxy-4-methyl-2(1H)-pyridinone; 2,2′-dipyridyl; 1,2-cyclohexanedione dioxime; 3-hydroxy-2-methyl-4-pyrone; 2,3-bis(2-pyridyl)-5,6-dihydropyrazine; 3-(4-phenyl-2-pyridyl)-5-phenyl-1,2,4-triazine; 5-hydroxy-2-(hydroxymethyl)-4H-pyran-4-one; 2,3-dihydroxypyridine; 2,2′-biquinoline; 2,2′-bipyrazine; 3-(2-pyridyl)-5,6-diphenyl-1,2,4-triazine; 4,4′-dimethyl-2,2′-dipyridyl; 4,5-dihydroxy-1,3-benzene-disulfonic acid; phenyl 2-pyridyl ketoxime; desferrioxamine B; 5,7-dichloro-8-hydroxyquinoline; 2,3-dihydroxynaphthalene; 2,3,5,6-tetrakis-(2′-pyridyl)pyrazine; 2,4-bis(5,6-diphenyl-1,2,4-triazine-3-yl)pyridine; di-2-pyridyl glyoxal; 2-(2-pyridyl)benzimidazole; 6-hydroxy-2-phenyl-3(2H)-pyridazinone; 2,4-pteridinediol; 3-(4-phenyl-2-pyridyl)-5,6-diphenyl-1,2,4-triazine; N-benzoyl-N-phenylhydroxylamine; 3-amino-5,6-dimethyl-1,2,4-triazine; 2,6-pyridinedicarboxylic acid; 2,4,5-trihydroxypyrimidine; 4-(2-amino-l-hydroxyethyl)-1,2-benzenediol; 3-hydroxy-5-(hydroxymethyl)-2-methyl-4-pyridine-carboxylic acid, phytic acid or mixtures thereof, including analogs, homologs or isomers of said chelating agents which themselves are chelating agents.

6. The method of claim 2 wherein from about 0.001 mg/cm² to about 1.0 mg/cm² of the chelating agent is applied to the skin.

7. The method of claim 2 wherein from about 0.01 mg/cm² to about 0.5 mg/cm² of the chelating agent is applied to skin.

8. The method of claim 2 wherein a safe and radioprotectively effective amount of an anti-inflammatory agent is also applied to the skin.

9. The method of claim 2 wherein a safe and radio-protectively effective amount of a radical scavenging compound is also applied to the skin.

10. A composition protective against damage to mammalian skin tissue by radiation treatment comprising:
(a) a safe and radioprotectively effective amount of a chelating agent; and
(b) a safe and effective amount of a carrier therefore.

11. A composition according to claim 10 adapted for topical application.

12. A composition according to claim 10 adapted for oral or intravenous administration.

13. The composition of claim 10 wherein said chelating agent is 2,2′-dipyridylamine; 1,10-phenanthroline; di-2-pyridylketone; 2-furildioxime; 2-furilmonoxime; 2,3-bis(2-pyridyl)pyrazine; 2,3-dihydroxybenzoic acid; ethylenediamine-N,N-bis(2-hydroxyphenylacetic acid), dimethyl ester; 1,1′-carbonyldiimidazole; 2,4,6-tri(2-pyridyl)-1,3,5-triazine; 2,2′-dipyridyi; 1,2-cyclohexanedione dioxime; 3-hydroxy-2-methyl-4-pyrone; 2,3-bis(2-pyridyl)-5,6-dihydropyrazine; 3-(4-phenyl-2-pyridyl)-5-phenyl-1,2,4-triazine; 2,3-dihydroxypyridine; 2,2′-biquinoline; 2,2′-bipyrazine; 3-(2-pyridyl)-5,6-diphenyl-1,2,4-triazine; 4,4′-dimethyl-2,2′-dipyridyl; 4,5-dihydroxy-1,3-benzene-disulfonic acid; phenyl 2-pyridyl ketoxime; desferrioxamine B; 5,7-dichloro-8-hydroxyquinoline; 2,3-dihydroxynaphthalene; 2,3,5,6-tetrakis-(2′-pyridyl)pyrazine; 2,4-bis(5,6-diphenyl-1,2,4-triazine-3-yl)pyridine; di-2-pyridyl glyoxal; 6-hydroxy-2-phenyl-3(2H)-pyridazinone; 2,4-pteridinediol; 3-(4-phenyl-2-pyridyl)-5,6-diphenyl-1,2,4-triazine; N-benzoyl-N-phenyl-hydroxylamine; 3-amino-5,6-dimethyl-1,2,4-triazine; 2,4,5-trihydroxypyrimidine; 4-(2-amino-1-hydroxyethyl)-1,2-benzenediol or mixtures thereof.

14. The composition of claim 10 wherein said chelating agent is 2,2′-dipyridylamine; 1,10-phenanthroline; di-2-pyridylketone; 2-furildioxime; 2-furilmonoxime; 2,3-bis(2-pyridyl) pyrazine; 2,3-dihydroxybenzoic acid; ethylenediamine-N,N-bis-(2-hydroxyphenylacetic acid) dimethyl ester; 1,1′-carbonyldiimidazole; 2,4,6-tri(2-pyridyl)-1,3,5-triazine or mixtures thereof.

15. The composition of claim 10 wherein said chelating agent is 2,2′-dipyridylamine; 1,10-phenanthroline; di-2-pyridylketone; 2-furildioxime; 2-furilmonoxime; 2,3-bis(2-pyridyl) pyrazine; [1-hydroxy-4-methyl-6-(2,4,4-trimethylpentyl)2(1H)-pyridone;]2,3-dihydroxybenzoic acid; ethylenediamine-N,N-bis-(2-hydroxyphenylacetic acid), dimethyl ester, phytic acid or mixtures thereof.

16. The composition of claim 10 wherein the chelating agent is 2-furildioxime.

17. The composition of claim 10 which comprises from about 0.01% to about 10% of the chelating agent.

18. The composition of claim 10 which additionally comprises a safe and radioprotectively effective amount of an anti-inflammatory agent.

19. The composition of claim 10 which additionally comprises a safe and radioprotectively effective amount of a radical scavenging compound.

20. An article of manufacture comprising packaging material and a chelating agent contained within said packaging material, wherein said chelating agent is effective for inhibiting the deleterious effects of radiation treatment therapy on mammalian skin tissue, and wherein said packaging material comprises a label which indicates that said chelating agent can be used for protecting against the deleterious effects of radiation treatment therapy on the skin.

21. An article of manufacture according to claim 20 wherein said chelating agent is 2,2′-dipyridylamine; 1,10-phenanthroline; di-2-pyridylketone; 2-furildioxime; 2-furilmonoxime; 2,3-bis(2-pyridyl)pyrazine; 2,3-dihydroxybenzoic acid; ethylenediamine-N,N-bis(2-hydroxyphenylacetic acid), dimethyl ester; 1,1′-carbonyldiimidazole; 2,4,6-tri(2-pyridyl)-1,3,5-triazine; 2,2′-dipyridyi; 1,2-cyclohexanedione dioxime; 3-hydroxy-2-methyl-4-pyrone; 2,3-bis(2-pyridyl)-5,6-dihydropyrazine; 3-(4-phenyl-2-pyridyl)-5-phenyl-1,2,4-triazine; 2,3-dihydroxypyridine; 2,2′-biquinoline; 2,2′-bipyrazine; 3-(2-pyridyl)-5,6-diphenyl-1,2,4-triazine; 4,4′-dimethyl-2,2′-dipyridyl; 4,5-dihydroxy-1,3-benzene-disulfonic acid; phenyl 2-pyridyl ketoxime; desferrioxamine B; 5,7-dichloro-8-hydroxyquinoline; 2,3-dihydroxynaphthalene; 2,3,5,6-tetrakis-(2′-pyridyl)pyrazine; 2,4-bis(5,6-diphenyl- 1,2,4-triazine-3-yl)pyridine; di-2-pyridyl glyoxal; 6-hydroxy-2-phenyl-3(2H)-pyridazinone; 2,4-pteridinediol; 3-(4-phenyl-2-pyridyl )-5,6-diphenyl-1,2,4-triazine; N-benzoyl-N-phenyl-hydroxylamine; 3-amino-5,6-dimethyl-1,2,4-triazine; 2,4,5-trihydroxypyrimidine; 4-(2-amino-1-hydroxyethyl)-1,2-benzenediol or mixtures thereof.

22. An article of manufacture according to claim 20 wherein said chelating agent is 2,2′-dipyridylamine; 1,10-phenanthroline; di-2-pyridylketone; 2-furildioxime; 2-furilmonoxime; 2,3-bis(2-pyridyl) pyrazine; 2,3-dihydroxybenzoic acid; ethylenediamine-N,N-bis-(2-hydroxyphenylacetic acid) dimethyl ester; 1,1′-carbonyldiimidazole; 2,4,6-tri(2-pyridyl)-1,3,5-triazine or mixtures thereof.

23. An article of manufacture according to claim 20 wherein said chelating agent is 2,2′-dipyridylamine; 1,10-phenanthroline; di-2-pyridylketone; 2-furildioxime; 2-furilmonoxime; 2,3-bis(2-pyridyl) pyrazine; [1-hydroxy-4-methyl-6-(2,4,4-trimethylpentyl)2(1H)-pyridone;] 2,3-dihydroxybenzoic acid; ethylenediamine-N,N-bis-(2-hydroxyphenylacetic acid), dimethyl ester, phytic acid or mixtures thereof.

24. An article of manufacture according to claim 20 wherein the chelating agent is 2-furildioxime.

25. An article of manufacture according to claim 20 which additionally comprises a safe and radioprotectively effective amount of an anti-inflammatory agent.

26. An article of manufacture according to claim 20 which additionally comprises a safe and radioprotectively effective amount of a radical scavenging compound.

27. An article of manufacture according to claim 20 wherein said label indicates that said chelating agent may be administered topically.

28. An article of manufacture according to claim 20 wherein said label indicates that said chelating agent may be administered orally or intravenously.
